(19) United States
(12) Patent Application Publication
(10) Pub. No.: US 2019/0385303 A1
(43) Pub. Date: Dec. 19, 2019

(54) METHOD AND COMPUTING DEVICE FOR EVALUATING THE QUALITY OF AN INSERTION OF AN ORTHOPEDIC IMPLANT

(71) Applicant: Medichanical Engineering ApS, Vejle (DK)

(72) Inventors: Eske Winther Petersen, Aalborg (DK); Poul Torben Nielsen, Aalborg (DK)

(21) Appl. No.: 16/009,864

(22) Filed: Jun. 15, 2018

(51) Int. Cl. G06T 7/00; A61B 6/03; A61B 6/12; A61B 5/055; A61B 5/00

(52) U.S. Cl. CPC G06T 7/0012; A61B 6/032; A61B 6/12; G06T 7/50; A61B 5/4851; G06T 2207/30052; A61B 5/055

(57) ABSTRACT

A post-surgery method for evaluating the quality of an insertion of an orthopedic implant (20) after surgery is provided. The method comprises the steps of determining at least two individual quality parameters (60) derived from at least one radiologic image (40) of the area comprising the orthopedic implant (20). The method further comprises the step of computing a total score (70) based on said at least two individual quality parameters (60), wherein the total score is indicative of the quality of the insertion of the orthopedic implant, and storing the total score.

### TECHNICAL FIELD OF THE INVENTION

The present invention relates to a method and a computing device for evaluating the quality of an insertion of an orthopedic implant.

### BACKGROUND

An orthopedic implant is a medical device for replacing a missing joint or bone or to support a damaged bone. A vast number of orthopedic implants for different parts of the human body exists, for example implants adapted for the hips, knees, shoulders, ankles, fingers, the spine etc.

Internal fixation is an operation in orthopedics that involves the surgical implementation of implants. Orthopedic implant surgery may use different kinds of techniques for attaching the implant to the bone. One example is to use bone cement to anchoring prosthesis components when inserting artificial joints. For implants being arranged with one or more sockets, the socket may be anchored in a cavity of the body (for example the pelvic cavity). For socket-implants the so-called “press-fit”-technique also exists, where a hole, is milled in the bone and a socket somewhat larger in diameter is subsequently inserted. A great tension is thereby achieved between bone and metal socket, whereby the metal socket is held in place without the need of screws.

Regardless of the fixation method used, there is a risk that the implant has not been optimally inserted and/or sized, since surgery is rather demanding and requires an experienced physician. If the implant is not securely clamped, there is a risk that it may loosen on either short- or long-term after the operation. If the implant is too strongly clamped, there is a substantial risk of fracture in the bone around the implant, whereby the implant becomes unstable.

Therefore, it is important to insert the right size of the implant. In order to determine which size of implant to be used, different measuring devices exists (for example a measuring head that can adopt several expanded states having different diameters, SE 1750690-8, for socket implants).

However, even if the right size of implant is chosen for the surgery, there is still a risk that the implant is placed incorrectly or not arranged in the most optimal way in the patient which may lead to e.g. increased wear of the implant and/or unwanted interference with human tissue.

An object of the present invention is to overcome one or more of the problems discussed above.

### SUMMARY OF THE INVENTION

In a first aspect, a post-surgery method for evaluating the quality of an insertion of an orthopedic implant is provided. The method comprises the steps of determining at least two individual quality parameters derived from at least one radiologic image of the area comprising the orthopedic implant, computing a total score based on said at least two individual quality parameters, wherein the total score is indicative of the quality of the insertion of the orthopedic implant, and storing the total score.

The step of determining the at least two individual quality parameters may be computed by a controller. The controller may be configured to determine the at least two individual quality parameters by calculating and/or measuring distances and/or angles in the at least one radiologic image of the area comprising the orthopedic implant and comparing the result to predetermined threshold values.

In one embodiment, the step of determining the at least two individual quality parameters is instead performed manually by calculating and/or measuring distances and/or angles in at least one radiologic image of the area comprising the orthopedic implant and comparing the result to predetermined threshold values.

The step of computing the total score may be performed by a controller. The controller may be configured to compute the total score as a weighted average of the at least two individual quality parameters.

The total score may be stored in a memory being in communication with the controller. The controller may be configured to compute statistical data of the total score and/or the at least two individual quality parameters over time, and save said data in the memory.

The individual quality parameters may relate to one or more of orientation, alignment, fixation, positioning, protrusion and diameter of the orthopedic implant.

In one embodiment, at least one of the individual quality parameters relate to the degree of protrusion of the orthopedic implant. The protrusion parameter may be given a score between a minimum score and a maximum score, wherein the maximum score will be awarded if the implant periphery protrudes less than a first threshold value and the minimum score will be awarded if the implant periphery protrudes more than a second threshold value. The first threshold value may be 5 degrees and the second threshold value may be 30 degrees. This is especially true for the acetabular component (cup) of a hip implant.

The at least one radiologic image may be an X-ray image, MRI image and/or a CT image.

The implant may comprise a first primary component and a second primary component and wherein the total score is a score for the first component, for the second component or a combination of both.

In one embodiment, the orthopedic implant is a hip implant. The implant may comprise an acetabular component and a femoral component and wherein the total score is a score for the acetabular component, for the femoral component or a combination of both.

In a second aspect a computing device for post-surgery evaluation of the quality of an insertion of an orthopedic implant is provided. The computer device comprises a controller being in communication with a memory, wherein the controller is configured to compute a total score based on at least two individual quality parameters derived from at least one radiologic image of the area comprising the orthopedic implant, wherein the total score is indicative of the quality of the insertion of the orthopedic implant, and store the total score in said memory.

The controller may further be configured to determine the at least two individual quality parameters derived from at least one radiologic image of the area comprising the orthopedic implant.

The controller may be configured to determine the at least two individual quality parameters by calculating and/or measuring distances and/or angles in the at least one radiologic image of the area comprising the orthopedic implant and comparing the result to predetermined threshold values.

The controller may be configured to compute the total score as a weighted average of the at least two individual quality parameters.

Generally, all terms used in the claims are to be interpreted according to their ordinary meaning in the technical field, unless explicitly defined otherwise herein. All references to “a/an/the [device, component, etc.]” are to be interpreted openly as referring to at least one instance of said device, component, etc., unless explicitly stated otherwise.

As used herein, the term “comprising” and variations of that term are not intended to exclude other components, integers, steps or materials.

Other objectives, features and advantages of the present invention will appear from the following detailed disclosure, from the attached claims, as well as from the drawings. It is noted that the invention relates to all possible combinations of features.

### BRIEF DESCRIPTION OF THE DRAWINGS

By way of example, embodiments of the present invention will now be described with reference to the accompanying drawings, in which:

FIG. 1 illustrates a post-surgery method of evaluating the quality of an insertion of an orthopedic implant according to one embodiment;

FIGS. 2a-b illustrate the method step of determining quality parameters and a total score according to one embodiment;

FIGS. 3a-e illustrate radiological images used to determine quality parameters according to one embodiment;

FIG. 4 illustrates a computing device according to one embodiment;

FIGS. 5a-b illustrate methods of evaluating the result of an orthopedic implant surgery according to different embodiments;

FIGS. 6a-b illustrate images used to determine quality parameters according to one embodiment; and

FIGS. 7a-b illustrate a display of a computing device according to FIG. 4.

### DETAILED DESCRIPTION OF PREFERRED EMBODIMENTS OF THE INVENTION

Orthopedic implant surgery may use different kinds of techniques for attaching the implant to the bone. All techniques have their own advantages and disadvantages, but none of the techniques can guarantee a perfect insertion of the implants. All patients and/or surgeons for all types of orthopedic implants would thus benefit of having a method or an apparatus for evaluating the result of an orthopedic implant surgery as will be described herein. More specifically, the method described herein is applicable for evaluating the quality of insertion of implants intended for hips, knees, shoulders, ankles, fingers, and the spine.

The fixation of the implant is associated with many different parameters that the surgeon needs to take into account in order to achieve the perfect fit of the implant to the bone. For the specific case of hip implants, some of the risks with a non-perfect fit are an early cup instability and/or dislocation, need of supplementary screws, cup deformation, increased wear of the cup, intraoperative occurrence of an acetabular fracture and implant interference such as iliopsoas impingement.

Moreover, the quality of the insertion may vary depending on the experience and knowledge of the surgeon. For the specific case of hip implants, there is a risk of a poor fixation and/or insufficient bone stuck causing aseptic loosening, cup oversizing causing pain and deformation, the use of a screw causing osteolysis, excessive retroversion or anteversion causing instability and impingement, and high scratch fit which increases the force required causing a pole gap or cup-protrusion.

Today no objective measurement of the quality of the insertion of the implant (such as for example the cup insertion of a hip implant) exists, instead the surgeon merely looks at the insertion while performing the surgery and in some cases looks at an X-ray image to visually determine that the insertion of the implant looks acceptable. Furthermore, there is no tool to objectively measure the quality of the surgeons and/or their improvements.

The inventors of the present invention have realized, after inventive reasoning, that there are a number of parameters that can help to evaluate the quality of the insertion of the implant into the bone. These parameters may relate to the orientation, the implant fixation, the implant diameter, the positioning of the implant and/or the protrusion of the implant. Any quality parameter that is performed less than perfect is correlated to an increased risk of complications and ultimately the risk of having a revision surgery. The quality parameters may then be used to generate a total quality assessment of the quality of the insertion of the implant. The quality can be defined as a measure of the risk of future complications.

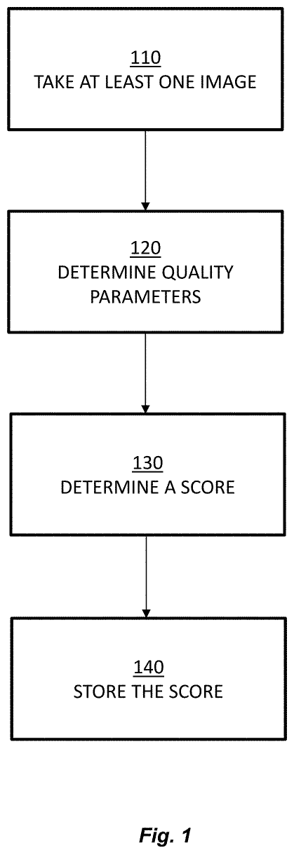

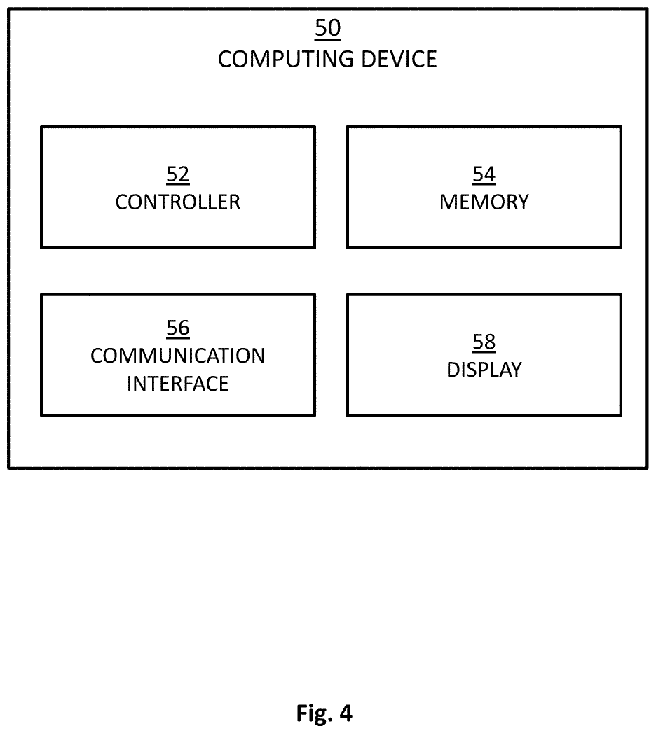

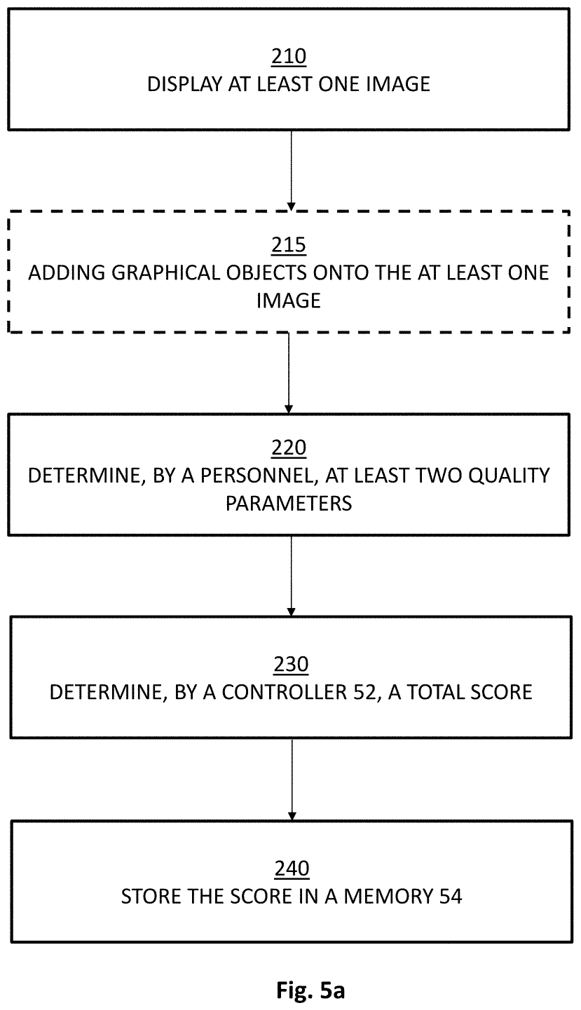

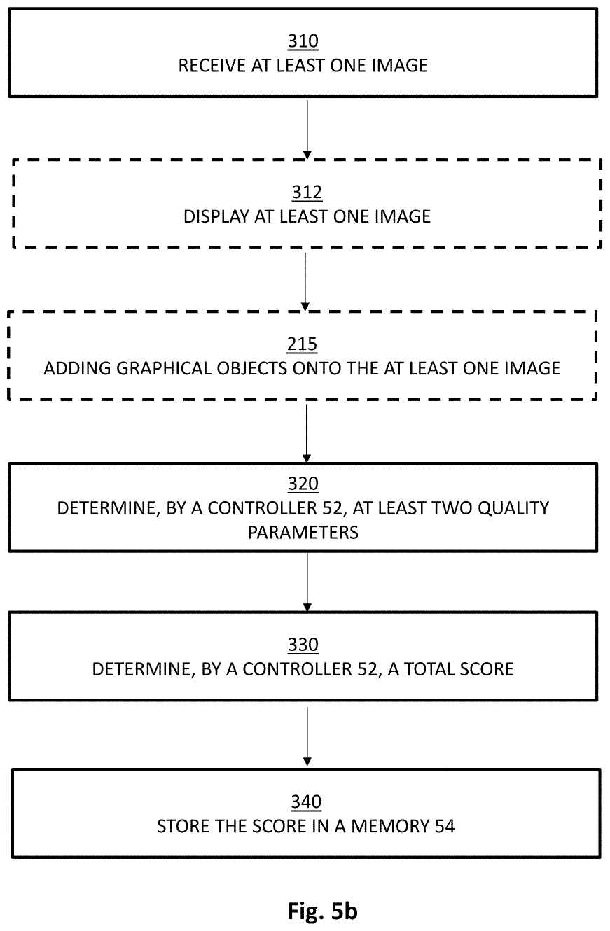

An embodiment of a method of evaluating the quality of an insertion of an orthopedic implant post-surgery is shown in FIG. 1. After the surgery is completed, at least one radiologic image is taken 110 of the area comprising the orthopedic implant. In a preferred embodiment, a number of radiological images are taken in different angles. The at least one radiologic image may be an X-ray image (potentially inclusive RSA), EOS scan, MRI-image and/or a CT-image. In one embodiment, the images are post-operative images of the area of the orthopedic implant. However, in some embodiments pre-operative images may also be used. If the implant is a hip implant, the radiologic images may for example be of a patient's pelvis and/or the femoral head of one of the hips. The at least one image may be displayed on the radiological image device itself and/or on a display 58 of a computing device 50, as will be described more with reference to FIGS. 4 and 5.

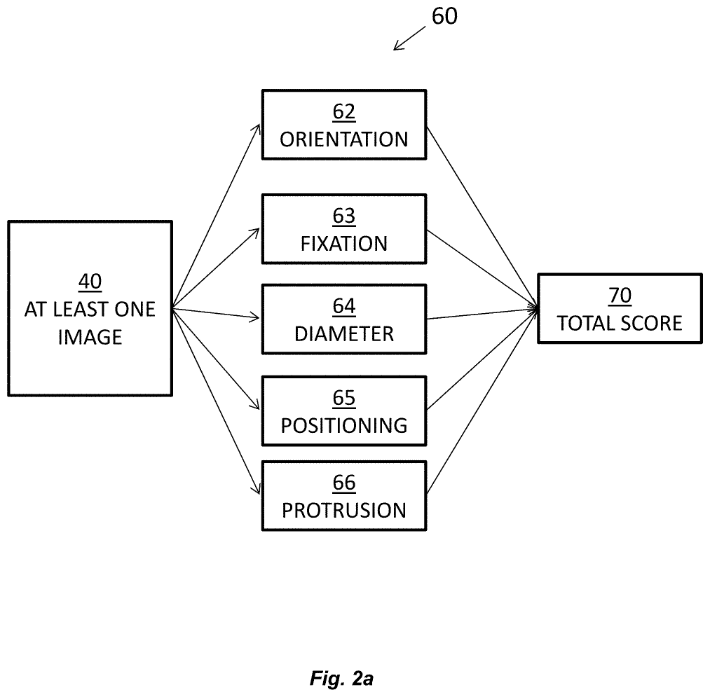

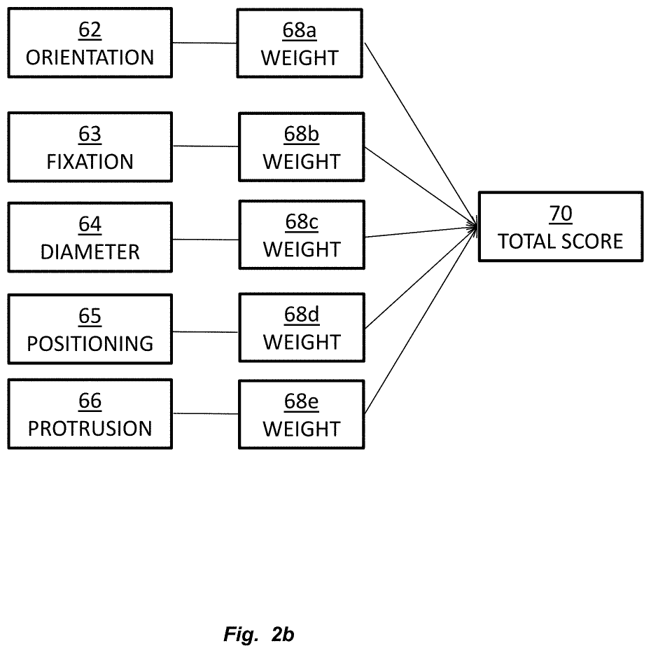

In a next step 120, at least two individual quality parameters are derived from the at least one radiologic image of the area comprising the orthopedic implant. The at least two individual quality parameters are determined by calculating distances and/or angles in the at least one radiologic image of the area comprising the orthopedic implant. This value(s) may then be compared predetermined threshold values and/or statistical data. The different quality parameters will be described more in reference to FIGS. 2a-b and 3a-e.

Based on at least two individual quality parameters, a total score is computed in a step 130. The total score is indicative of the quality of the insertion of the implant that was performed during the surgery. In one embodiment, the total score is a value between 0 and 10, where 10 is the highest score, i.e. best score, and 0 is the lowest score. However, the total score could be of any other type of range, such as 0-5, 0-50, 0-100 or any other suitable range. The total score is then saved in a step 140.

The total score may be computed for the whole implant, and/or for some parts of the implant. In one embodiment, the implant comprises a first primary component and a second primary component. For a hip implant, the primary component may be the acetabular component (the cup) and the second primary component may be the femoral component (the stem). In one embodiment, different quality parameters are used for the first and second component. In an alternative embodiment, the same quality parameters are used for both the first and the second component. The total score may be a score computed for the first component, for the second component or a combination of both. Hence, the total score may be a total score of the whole implant or some parts of the implant.

In one embodiment, values of a set of quality parameters are determined for the first component. These values are then used to compute a total sub-score for the first component. Before, after or simultaneously, a set of quality parameters are determined for the second component. These values are then used to compute a total sub-score for the second component. In a further step, the two total sub-scores are combined in order to generate a final total score for the quality of the insertion of the implant.

In some embodiment, the total score is computed using the total sub-scores. When doing this combination, a further quality parameter 60 may be check to ensure that the first and second components are matched properly. Depending on the severity of such a possible mismatch, X points should be deducted from the total score. In one example, the first component is a cup and the second component is a stem and the two parts are matched (caput vs. liner match) so see that the cup and stem match each other properly.

In yet one embodiment, quality parameters are determined for the first and second component. These quality parameters are then used to compute the total score for the quality of the insertion of the implant. In this embodiment, no total sub-score is computed.

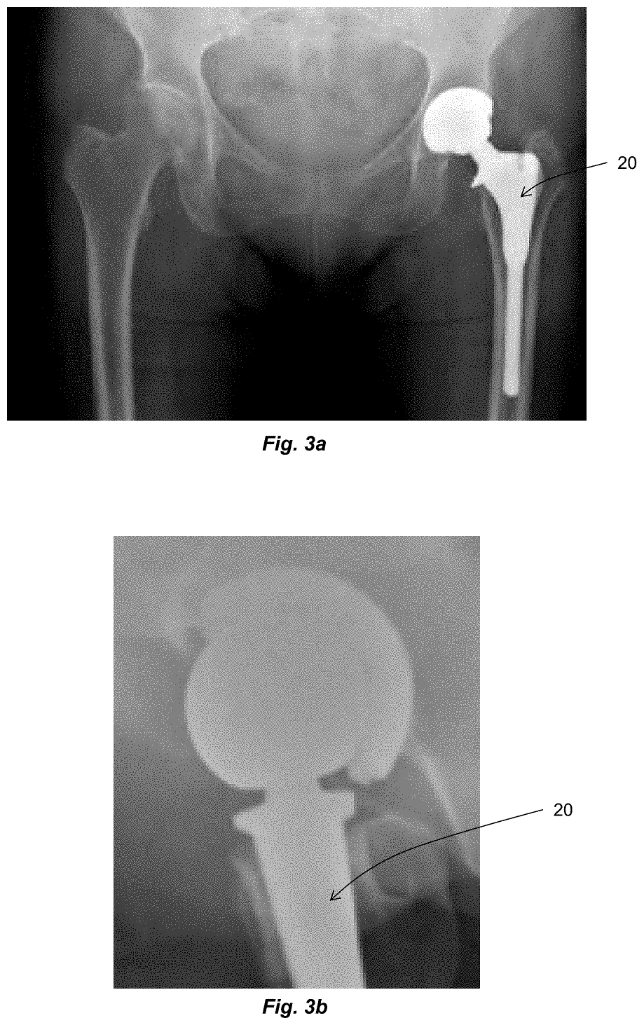

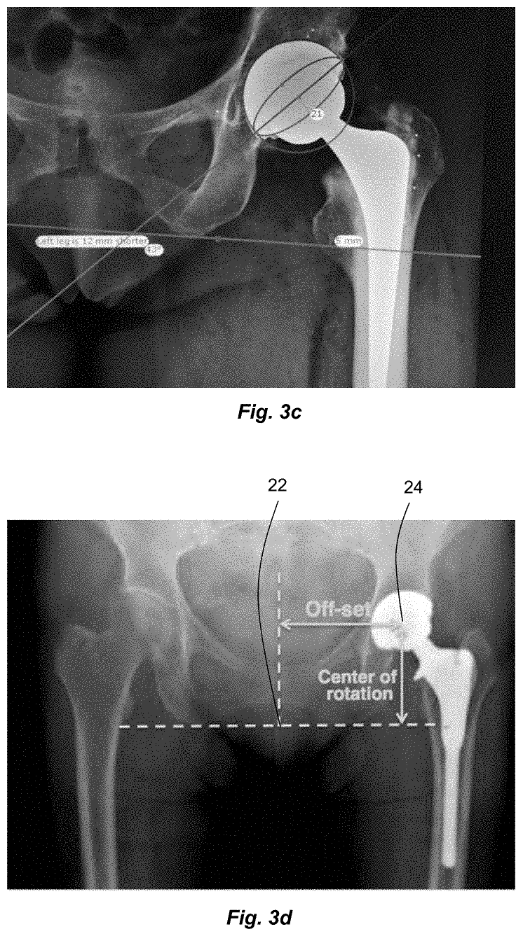

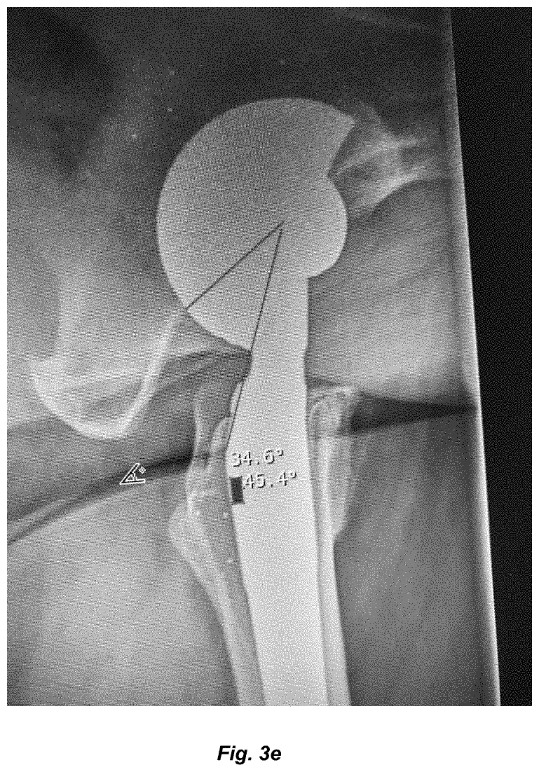

FIG. 2a illustrates a number of different individual quality parameters 60 that may be used to compute the total score 70 in a step 130. The individual quality parameters 60 may for example be related to orientation 62, fixation 63, positioning 65, protrusion 66 and/or diameter 64 of the orthopedic implant. Details on these individual quality parameters 60 will be described with reference to FIGS. 3a-e. As should be understood by a person skilled in the art other parameters could also be of relevance. Although the description is mainly focused on implants for hips, the quality parameters could also be used for example knees, shoulders, ankles, fingers, and the spine. Depending on the type of implant other quality parameters may also be of relevance.

Each individual parameter 60 is assigned a value depending on the how well the implant in the at least one image 40 satisfy that specific parameter. The value may for example range between 0 and 10, where 10 is the highest value, i.e. best value, and 0 is the lowest value. However, the value could be of any other type of range, such as 0-5, 0-50, 0-100 or any other suitable range.

Once the scores of the individual quality parameters 60 have been determined, the total score 70 is to be computed. The different individual quality parameters 60 may have different significance of the result of the implant surgery. Hence, some parameters 60 may be more important than others when determining the total score 70. Moreover, some parameters 60 may be linked to each other so that a synergistic effect is caused. In one embodiment, at least one of the different individual quality parameters are thus weighted 68a-e when computing the total score 70. This is schematically illustrated in FIG. 2b. The weight parameters 68a-e of the individual quality parameters 60 may be different from each other, and/or some weight parameters 68a-e may be the same.

In one embodiment, the weighting 68a-e of the scores of the individual quality parameters are determined by using a population of historical x-rays where clinical follow-up data from the relevant patients are available. The clinical results, such as failures (e.g. dislocation, pain, implant wear, osseolysis (bone loss) etc.), are correlated to the awarded scores. Based on statistical data it is determined which parameters should be ranked as the most important, i.e. gaining the highest weight factor, and so on.

In one embodiment, the total score 70 is determined by having a maximum score for each individual parameter 60. As an example the following weight conditions 68a-e may be used when computing the total score for the first component of a hip-implant (being the cup); orientation 62 can account for maximum 4 points of the total cup-score 70, fixation 63 can account for maximum 2 points, diameter 64 can account for maximum 1 point, positioning 65 can account for maximum 1 point, protrusion 66 can account for maximum 2 points, and if the cup is dislocated the total score will be 0. Hence, the total score may never be above 0 if the cup is dislocated. Moreover, in this example the orientation 62 is a more important parameter 60 when computing the total score 70 than for example the parameters for positioning 65 and/or diameter 64. As should be understood by a person skilled in the art, other weighting conditions 68a-e could be used. Moreover, although the example was focused on only one component of the implant, the same reasoning applies for the second component and the whole implant.

The total score 70 may thus be computed as a weighted average of the scores of the individual quality parameters 60. Weighted average is a mean calculated by giving values in a data set more or less influence according to some attribute of the data.

If a total sub-score for the first component and the second component is computed, these two sub-scores may be combined to the total score by applying weighting conditions.

As previously stated some parameters 60 may be linked to each other causing a synergistic effect. One example, is that a too large cup diameter is likely to also mean increased protrusion. It is also possible that e.g. two otherwise equally ranked parameters that are both sub-optimal will amplify their impact on the risk of clinical complications. In these cases, these parameters could for example be multiplied by a factor if they are both under a certain threshold.

The different parameters will now be described with reference to FIG. 3-e showing images 40 of an implant 20. The examples presented here after are mainly related to the first component of an implant, and more specifically to the first component of a hip implant. However, it should be understood that the following also could be applied to other types of implants as well as the second component of the hip implant and the hip-implant as a whole.

Cup Fixation 63

When determining the cup fixation 63, it is preferred if two radiological images 40 are used. In one embodiment, an anteroposterior x-ray (as in FIG. 3a) and axial x-ray (as seen in FIG. 3b) are used. In order to determine the quality of the cup fixation 63, visible signs of pen-prosthetic fractures are analysed based on the one or more post-operative image(s) 40. Based on the at least one image, preferably two images, the cup dislocation is determined. Additionally, it can be determined if any screws are used to fixate the implant. Hence, the parameter of cup fixation 63 may be seen as comprising two sub-parameters being dislocation and the use of screws.

In one embodiment, the cup fixation 63 is awarded a score on a scale from 0 to 10. The maximum score, i.e. 10, will be awarded if there is no visible fracture, no cup dislocation and no screws. The minimum score, i.e. 0, will be awarded if the cup is dislocated. A number X of points are deducted from the maximum score if fracture(s) are detected, where the number X depends on the location and severity of the fracture(s). Additionally, a number Y of points are deducted from the maximum score if screws are used. The number Y may for example be determined by analysis of historic cohort. The numbers X and Y may be any number between 10 and 1.

Cup Diameter vs. Caput Diameter 64

When determining the quality parameter related to the cup diameter 64 and the caput diameter, it is preferred if two radiological images 40 are used. In one embodiment, a post-operative image of the operated hip is used. Additionally, it is beneficial if a pre-operative image of the femoral head of the relevant hip and/or a suitable image of the femoral head of the opposite non-operated hip is used.

In order to determine the quality of the cup diameter 64, the cup-diameter is measured and compared with the diameter of the femoral head of pre-operative image or with the opposite non-operated hip.

In one embodiment, the cup diameter 64 is awarded a score on a scale from 0 to 10. The maximum score, i.e. 10, will be awarded if the diameter of the cup is below a first threshold value. The first threshold value may for example be if the cup diameter is 6 mm or less than 6 mm larger than the femoral head (caput) diameter. The minimum score, i.e. 0, will be awarded if the cup diameter is above a second threshold value. The second threshold value may for example be if the cup diameter is more than 12 mm larger than the femoral head (caput) diameter. As should be understood by the person skilled in the art, other thresholds may be used. For example the score of 5 may be awarded if the diameter of the cup is between the first threshold value and the second threshold value. In this example a score of 5 is awarded if the diameter is more than 6 mm and less than 12 mm larger than the femoral head diameter.

Positioning 65

When determining the quality parameter related to the cup position 65, it is preferred if two radiological images are used. In one embodiment, a post-operative image of the operated hip is used. Additionally, it is beneficial if a pre-operative image of femoral head of relevant hip and/or a suitable image of femoral head of opposite non-operated hip is used. In one embodiment, a post-operative image of the operated hip is used. Additionally, it is beneficial if a pre-operative image of the relevant hip and/or a suitable image of the opposite non-operated hip is used.

In order to determine the quality of the cup position 65, at least one distance from at least one selected anatomical landmark 22 to the centre 24 of the joint is measured. This is illustrated in FIG. 3d. The at least one distance is compared to the same distance in a pre-operative image and/or compared to the opposite non-operated hip. Distances may be measured relative to horizontal/vertical lines defined by anatomical landmarks.

In one embodiment, the cup positioning 65 is awarded a score on a scale from 0 to 10. The maximum score, i.e. 10, will be awarded if the centre of the joint is positioned below a first threshold value from the original joint or mirrored from the centre of the opposite non-operated joint. The first threshold value may for example be if the centre joint is less than 5 mm away from the original joint or mirrored from the centre of the opposite non-operated joint. The minimum score, i.e. 0, will be awarded if the centre of the joint is positioned more than a second threshold value from the original joint or mirrored from the centre of the opposite non-operated joint. The second threshold value may for example be if the centre joint is more than 10 mm away from the original joint or mirrored from the centre of the opposite non-operated joint. As should be understood by the person skilled in the art, other additional thresholds may be used. For example the score of 5 may be awarded if the centre of the joint is positioned between the first threshold value and the second threshold value. In this example a score of 5 is awarded if the centre joint is between 5 mm and 10 mm away from the original joint or mirrored from the centre of the opposite non-operated joint.

Protrusion 66

When determining the cup protrusion 66, it is preferred if two radiological images 40 are used. In one embodiment, an anteroposterior x-ray (as in FIG. 3a) and an axial x-ray (as seen in FIG. 3b) are used. In order to determine the quality of the cup protrusion, the cup periphery that protrudes outside the acetabulum, in e.g. anteroposterior and axial images, is measured. This can be measured in degrees, millimetres and/or shares of the cup. In the embodiment shown in FIG. 3e, the protrusion 66 is measured in degrees.

In one embodiment, the cup protrusion 66 is awarded a score on a scale from 0 to 10. The maximum score, i.e. 10, will be awarded if the cup periphery protrudes less than a first threshold value outside the acetabulum. The first threshold value may be 5 degrees. The minimum score, i.e. 0, will be awarded if the cups periphery protrudes more than a second threshold value outside the acetabulum. The second threshold value may be 30 degrees.

As should be understood by the person skilled in the art, other additional thresholds may be used. For example the score of 5 may be awarded if the cup periphery protrudes between the first threshold and the second threshold. In this example a score of 5 is awarded if the cup periphery protrudes outside the acetabulum by 5 to 30 degrees. In yet one example, the score 9 is awarded if the cup periphery protrudes between 5 and 7 degrees outside the acetabulum, and a score 8 is awarded if the cup periphery protrudes between 7.1 and 10 degrees outside the acetabulum, a score 7 is awarded if the cup periphery protrudes between 10.1 and 12 degrees outside the acetabulum, and so on.

Cup Orientation 62

When determining the cup orientation 62, it is preferred if two radiological images 40 are used. In one embodiment, an anteroposterior x-ray (as in FIG. 3a) and an axial x-ray (as seen in FIG. 3b) are used. In order to determine the quality of the cup orientation 62, the degree of cup inclination relative to a horizontal line on an image is measured. Additionally, it is determined if the cup is anteverted or retroverted. This is preferably determined using the axial image, as is shown in FIG. 3c. Moreover, the degrees of anteversion/retroversion is measured on the anteroposterior image.

The cup orientation 62 value may comprise two sub-parameters. The sub-parameters may be inclination and anteversion/retroversion. In one embodiment, the cup orientation is awarded a score on a scale from 0 to 10. Each parameter can be awarded a subscore. The two subscores are then combined, for example the average of both subscores, in order to generate the cup orientation score. The maximum score, i.e. 10, will be awarded if the average of the subscores for inclination and anteversion/retroversion result in the maximum score.

The subscore for inclination will be maximum if the inclination relative to horizontal line is within a given first range. The first range may for example be 30-45 degrees. The subscore for inclination will be minimum if the inclination relative to horizontal is in a given second range. The range may also be seen as being below a first threshold or above a second threshold. The second range may for example be less than 20 degrees or more than 60 degrees, i.e. the minimum value will be awarded if the inclination is less than 20 degrees or higher than 60 degrees.

The subscore for anteversion/retroversion will be maximum if the degree of anteversion/retroversion is in a given first range. The first range may for example be 15-25 degrees. The subscore for anteversion/retroversion will be maximum if the degree of anteversion/retroversion is in a given second interval. The range may also be seen as being below a first threshold or above a second threshold. The second interval may for example be less than 0 degrees or more than 40 degrees, i.e. the minimum value will be awarded if the degree of anteversion/retroversion is less than 0 degrees or higher than 40 degrees.

Cement/Bone Interdigitating

In situations where bone cement is used for fixation, a quality parameter could be cement/bone interdigitating (cement penetration into cancellous bone). The cement/bone interdigitating comprise two sub-parameters. The sub-parameters may be cement/bone interdigitating and thickness of the cement mantle.

In one embodiment, the cement/bone interdigitating is awarded a sub-score where the maximum score will be awarded if white-out is detected The minimum score will be awarded if radiolucency between bone and cement is detected.

The thickness of the cement mantle is awarded a sub-score where the maximum score is awarded for a thickness above 2 mm and a minimum score is awarded if between 0 to 1 mm.

Other quality parameters 60 that could be measured are alignment, rotation, center of rotation, signs of fracture and presence of bone cement. These quality parameters 60 are especially beneficial for the second component of the implant, being the component that is elongated. For the example where the second component is the femoral component of a hip (the stem) the following features can be measured being related to alignment relative to the femoral axis, rotation in relation to the femoral neck (anteversion, retroversion), centre of rotation, signs of fracture and signs of cement.

For the example when the implant is a knee implant, the following quality parameters are especially useful: femoral component and tibial component positioning compared to ideal, knee axis, liner fixation (dislocation), implant sizing in relation to ideal (may depend on implant type), the use of bone cement. As previously stated, the total score 60 may be computed based on any one of at least two quality parameters.

For the example when the implant is intended for a shoulder, ankle, elbow, wrist, finger etc., parameters that are especially useful are: alignment/positioning relative to anatomical landmarks, implant sizing compared to ideal values, liner dislocation, fracture(s), and the use of cement.

For the example of implants relating to the spine, the most relevant quality parameters may be screw malpositioning and restoration of anatomical measures/curves etc. The method of evaluating the quality of an insertion of an implant, i.e. the result of an orthopedic implant surgery, as shown in FIG. 1 and FIGS. 4a-b will now be described more in detail.

In a first embodiment, the method according to FIG. 1 is performed manually by trained personnel. Hence, after the surgery is completed and the at least one radiologic image 40 is taken, the at least two individual quality parameters are derived, by trained personnel, from the at least one radiologic image 40 of the area comprising the orthopedic implant. The personnel looks at the image(s) 40 and measures distances and/or angles in the at least one radiologic image of the area comprising the orthopedic implant. The value(s) are then compared against predetermined threshold values, for example using a stored table. Based on at least two individual quality parameters, a total score is computed in step 130 by combining the different parameters, possibly using weights, as previously described. Once the personnel has computed the total score, that score is saved for example in the patients file or in a database.

In an alternative embodiment, at least some parts of the method according to FIG. 1 is performed using a computing device 50 as illustrated in FIG. 4. The computing device 50 comprises a controller 52 and an associated memory 54.

The controller 52 is responsible for the overall operation of the computing device 50 and is preferably implemented by any commercially available CPU (“Central Processing Unit”), DSP (“Digital Signal Processor”) or any other electronic programmable logic device. The controller 52 is configured to read instructions from the memory 54 and execute these instructions to control the operation of the computing device 50. The memory 54 may be implemented using any commonly known technology for computer-readable memories such as ROM, RAM, SRAM, DRAM, CMOS, FLASH, DDR, SDRAM or some other memory technology. The memory 54 is used for various purposes by the controller 52, one of them being for storing application data and program instructions for various software modules in the computing device 50.

The computing device 50 may further comprise a communication interface 56 for connecting with other devices. Such interfaces may be wired or wireless. Some examples of interfaces are USB (Universal Serial Bus) ports, Bluetooth™ ports, Ethernet ports or WiFi (according to IEEE standard 802.11) ports. In one embodiment the communication interface 56 is a wired connection. The communication interface 56 may for example be arranged to receive the at least one image 40.

Additionally, the computing device 50 may comprise a display 58. In one embodiment the display 58 is a touch display. In other embodiments the display 58 is a non-touch display. The computing device 50 may further comprise one or a plurality of physical keys to control the operation of the computing device 50.

In one embodiment, as shown in FIG. 5a, the computing device 50 is configured to receive at least one radiologic image 40. The computing device 50 may receive the image(s) by the communication interface 56. The image(s) 40 may then be displayed on the display 58 of the computing device 50.

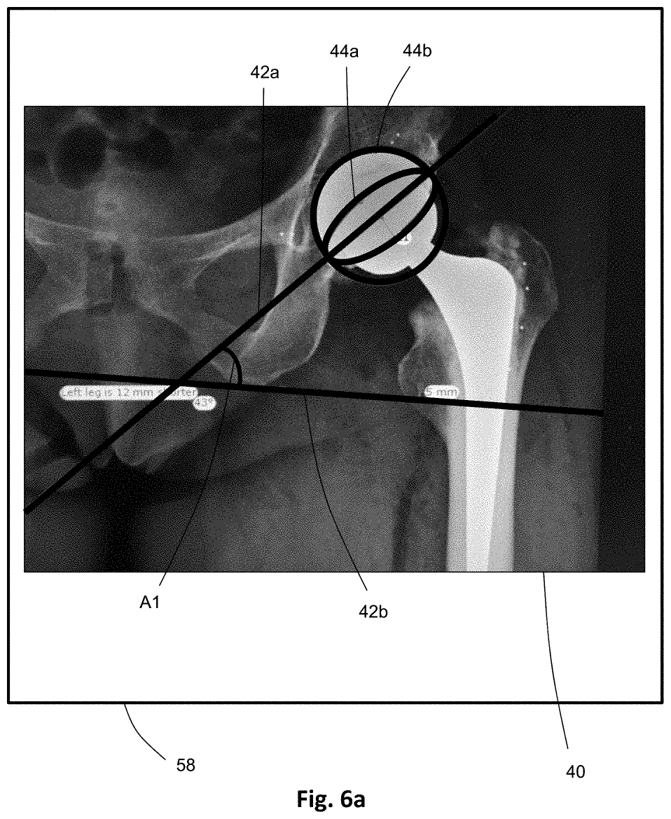

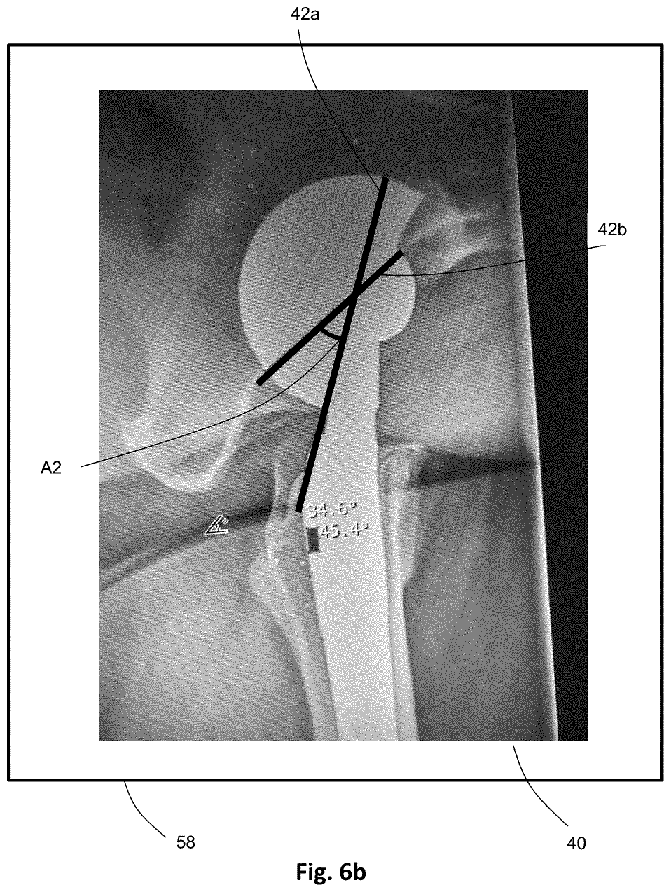

The trained personnel looks at the image(s) 40 displayed on the computing device 50 and calculates values of the at least two individual quality parameters based on the image(s). The computing device 50 may be arranged to display one or more graphical objects 42a-b, 44a-b (as seen in FIGS. 6a and b) in the image(s) 40 being shown on the display 58. The graphical objects 42a-b, 44a-b may be used by the personnel to draw lines 42a-b, circles 44a-b, angles or similar objects that could be used to assist in determining the individual quality parameters. The graphical objects 42a-b, 44a-b may be automatically added by the controller 52, or manually added by the personnel.

The personnel then enters the values of the calculated quality parameters into the computing device 50. Based on the entered quality parameters, the controller 52 of the computing device 50 computes a total score. The controller 52 may compute the total score by applying a weight to one or more of the individual quality parameters. Once the total score has been computed by the controller 52, it is saved in the associated memory 54.

FIG. 5b illustrates an alternative embodiment where the steps of receiving 310 at least one image, determining 320 at least two quality parameters, determining 330 a total score and saving 340 the total score are performed by the controller 52 of the computing device 50.

The computing device 50 is configured to receive the image(s), for example by the communication interface 56. The image(s) 40 may then be displayed on the display 58 of the computing device 50. In some embodiment, the display also displays graphical objects 42a-b, 44a-b that can be added, either by the personnel or by the computing device itself, to the image displayed on the display.

The controller 52 is configured to determine at least two individual quality parameters based on the image(s). This may be done completely automatic or semi-automatic, as will now be described. In one embodiment, image recognition algorithms of the computing device 50 are arranged to recognize specific points and/or lines in the image. Based on those points the controller 52 may be configured to derive quality parameters.

In one embodiment, the personnel adds graphical objects 42a-b, 44a-b in the image(s) displayed on the display, marking up specific lines and/or points. The controller 52 is then configured to derive quality parameters based on these points and/or lines. This is further illustrated in FIGS. 6a-b, showing a display 58 displaying an image 40 having a plurality of graphical objects 42a-b, 44a-b arranged in an overlying layer.

In FIG. 6a, the radiological image 40 is arranged with two graphical lines 42a, 42b with a specified angle A1 between the lines. Moreover, the image 40 is arranged with two circles 44a, 44b arranged to represent the spherical part of the implant. In this specific example, the orientation of the implant is evaluated. However, depending on what quality parameter is computed, different graphical objects are added to the image. FIG. 6b, illustrates a radiological image 40 arranged with two graphical lines 42a, 42b with a specified angle A2 between the lines. In this specific example, the quality parameter that is computed is the protrusion of the implant.

In a preferred embodiment, the values of the individual quality parameters are stored in the memory 54. The determined values of the quality parameters are then used to compute, by the controller 52, a total score. The controller 52 may compute the total score by applying a weight to one or more of the individual quality parameters. Once the total score is computed by the controller 52, it is saved in the associated memory 54.

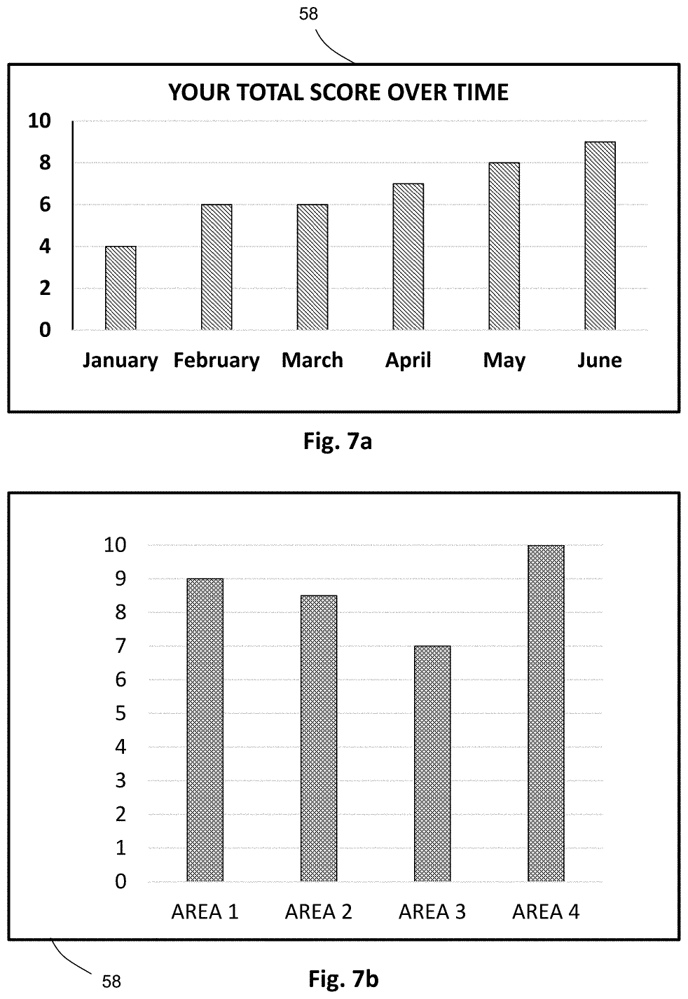

The total score and/or the individual quality parameters could be stored in the associated memory 54. The stored values could be processed, by the controller 52, to generate different kinds of statistical data. The statistical data is preferably displayed on the display 58. Some examples of statistical data are shown in FIGS. 7a-b.

FIG. 7a illustrates an example of statistical data saved for each surgeon, where the total score is shown over time. In this example, the total score is shown for the last six months. As should be understood by a person skilled in the art, the statistic could also show for one or a plurality of specific individual quality parameter(s) and/or over different time spans.

FIG. 7b illustrates an example of statistical data where all surgeons for one hospital (in the figure marked as areas) are added together into one column. It is thus possible to compare the quality of the surgeons at different hospitals. As should be understood by a person skilled in art, the statistics could also be shown for one specific hospital comparing the different surgeons working therein. Statistics computed by the controller may be useful in order to generate feedback to the individual surgeon, feedback for the hospital manager and/or feedback to politicians or other decision-makers. Statistics related to the quality of the surgical procedures may be used to conserve and optimize resources such as the number of new surgeons attending to a specific hospital and/or the amount of additional education needed at some specific hospitals.

The controller 52 may be configured to compute different values from the individual quality parameters and/or the total score. In one embodiment, the average value of the total score, during a given time span, is computed. Additionally, or alternatively, the individual quality parameter that has been improved the most, during a given time span, is computed. Additionally, or alternatively, the individual quality parameter that has the highest improvement potential is computed. The given time span may be given in days, weeks, months, years or in number of surgical procedures.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Aspects

In view of the described methods and devices and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A post-surgery method for evaluating the quality of an insertion of an orthopedic implant (20), wherein the method comprises the steps of:

determining at least two individual quality parameters (60) derived from at least one radiologic image (40) of the area comprising the orthopedic implant (20);

computing a total score (70) based on said at least two individual quality parameters (60), wherein the total score is indicative of the quality of the insertion of the orthopedic implant; and

storing the total score.

Aspect 2: The method according to aspect 1, wherein the step of determining the at least two individual quality parameters (60) is computed by a controller (52).

Aspect 3: The method according to aspect 2, wherein the controller (52) is configured to determine the at least two individual quality parameters (60) by calculating and/or measuring distances and/or angles in the at least one radiologic image (40) of the area comprising the orthopedic implant (20) and comparing the result to predetermined threshold values.

Aspect 4: The method according to aspect 1, wherein the step of determining the at least two individual quality parameters (60) is performed manually by calculating and/or measuring distances and/or angles in at least one radiologic image (40) of the area comprising the orthopedic implant (20) and comparing the result to predetermined threshold values.

Aspect 5: The method according to any preceding aspects, wherein the step of computing the total score (70) is performed by a controller (52).

Aspect 6: The method according to aspect 5, wherein the controller (52) is configured to compute the total score (70) as a weighted average of the at least two individual quality parameters (60).

Aspect 7: The method according to aspect 5 or 6, wherein the total score (70) is stored in a memory (54) being in communication with the controller (52).

Aspect 8: The method according to aspect 7, wherein the controller (52) is configured to compute statistical data of the total score (70) and/or the at least two individual quality parameters (60) over time, and save said data in the memory (54).

Aspect 9: The method according to any preceding aspects, wherein the individual quality parameters (60) relate(s) to one or more of orientation (62), fixation (63), alignment, positioning (64), protrusion (66) and diameter (64) of the orthopedic implant (20).

Aspect 10: The method according to any preceding aspects, wherein at least one of the individual quality parameters (60) relate to the degree of protrusion (66) of the orthopedic implant.

Aspect 11: The method according to aspect 10, wherein the the protrusion parameter is given a score between a minimum score and a maximum score, wherein the maximum score will be awarded if the implant periphery protrudes less than a first threshold value and the minimum score will be awarded if the implant periphery protrudes more than a second threshold value.

Aspect 12: The method according to aspect 11, wherein the first threshold value is 5 degrees and the second threshold value is 30 degrees.

Aspect 13: The method according to any preceding aspects, wherein the at least one radiologic image is an X-ray image, MRI image and/or a CT image.

Aspect 14: The method according to any preceding aspects, wherein the implant comprises a first primary component and a second primary component and wherein the total score (70) is a score for the first component, for the second component or a combination of both.

Aspect 15: The method according to any preceding aspects, wherein the orthopedic implant (20) is a hip implant.

Aspect 16: The method according to aspect 15, wherein the implant comprises an acetabular component and a femoral component and wherein the total score (70) is a score for the acetabular component, for the femoral component or a combination of both.

Aspect 17: A computing device (50) for post-surgery evaluation of the quality of an insertion of an orthopedic implant (20), wherein the computer device (50) comprises a controller (52) being in communication with a memory (54), wherein the controller (52) is configured to:

compute a total score (70) based on at least two individual quality parameters (60) derived from at least one radiologic image (40) of the area comprising the orthopedic implant (20), wherein the total score (70) is indicative of the quality of the insertion of the orthopedic implant; and

store the total score in said memory (54).

Aspect 18: The computing device (50) according to aspect 17, wherein the controller (52) is further configured to determine the at least two individual quality parameters (60) derived from at least one radiologic image (40) of the area comprising the orthopedic implant (20).

Aspect 19: The computing device (50) according to aspect 18, wherein the controller (52) is configured to determine the at least two individual quality parameters (60) by calculating and/or measuring distances and/or angles in the at least one radiologic image (40) of the area comprising the orthopedic implant (20) and comparing the result to predetermined threshold values.

Aspect 20: The computing device (50) according to any one of aspect 17 to 19, wherein the controller (52) is configured to compute the total score (70) as a weighted average of the at least two individual quality parameters (60).

1. A post-surgery method for evaluating the quality of an insertion of an orthopedic implant, wherein the method comprises the steps of:
determining at least two individual quality parameters derived from at least one radiologic image of the area comprising the orthopedic implant;
computing a total score based on said at least two individual quality parameters, wherein the total score is indicative of the quality of the insertion of the orthopedic implant; and
storing the total score.

2. The method according to claim 1, wherein the step of determining the at least two individual quality parameters is computed by a controller.

3. The method according to claim 2, wherein the controller is configured to determine the at least two individual quality parameters by calculating and/or measuring distances and/or angles in the at least one radiologic image of the area comprising the orthopedic implant and comparing the result to predetermined threshold values.

4. The method according to claim 1, wherein the step of determining the at least two individual quality parameters is performed manually by calculating and/or measuring distances and/or angles in at least one radiologic image of the area comprising the orthopedic implant and comparing the result to predetermined threshold values.

5. The method according to claim 1, wherein the step of computing the total score is performed by a controller.

6. The method according to claim 5, wherein the controller is configured to compute the total score as a weighted average of the at least two individual quality parameters.

7. The method according to claim 5, wherein the total score is stored in a memory being in communication with the controller.

8. The method according to claim 7, wherein the controller is configured to compute statistical data of the total score and/or the at least two individual quality parameters over time, and save said data in the memory.

9. The method according to claim 1, wherein the individual quality parameters relate(s) to one or more of orientation, fixation, positioning, protrusion and diameter of the orthopedic implant.

10. The method according to claim 1, wherein at least one of the individual quality parameters relate to the degree of protrusion of the orthopedic implant.

11. The method according to claim 10, wherein the the protrusion parameter is given a score between a minimum score and a maximum score, wherein the maximum score will be awarded if the implant periphery protrudes less than a first threshold value and the minimum score will be awarded if the implant periphery protrudes more than a second threshold value.

12. The method according to claim 11, wherein the first threshold value is 5 degrees and the second threshold value is 30 degrees.

13. The method according to claim 1, wherein the at least one radiologic image is an X-ray image, MRI image and/or a CT image.

14. The method according to claim 1, wherein the implant comprises a first primary component and a second primary component and wherein the total score is a score for the first component, for the second component or a combination of both.

15. The method according to claim 1, wherein the orthopedic implant is a hip implant.

16. The method according to claim 15, wherein the implant comprises an acetabular component and a femoral component and wherein the total score is a score for the acetabular component, for the femoral component or a combination of both.

17. A computing device for post-surgery evaluation of the quality of an insertion of an orthopedic implant, wherein the computer device comprises a controller being in communication with a memory, wherein the controller is configured to:
compute a total score based on at least two individual quality parameters derived from at least one radiologic image of the area comprising the orthopedic implant, wherein the total score is indicative of the quality of the insertion of the orthopedic implant; and
store the total score in said memory.

18. The computing device according to claim 17, wherein the controller is further configured to determine the at least two individual quality parameters derived from at least one radiologic image of the area comprising the orthopedic implant.

19. The computing device according to claim 18, wherein the controller is configured to determine the at least two individual quality parameters by calculating and/or measuring distances and/or angles in the at least one radiologic image of the area comprising the orthopedic implant and comparing the result to predetermined threshold values.

20. The computing device according to claim 17, wherein the controller is configured to compute the total score as a weighted average of the at least two individual quality parameters.
